(19) United States
(12) Patent Application Publication
(10) Pub. No.: US 2025/0245821 A1
(43) Pub. Date: Jul. 31, 2025

(54) ENDOSCOPE APPARATUS, OPERATION METHOD OF ENDOSCOPE APPARATUS, AND STORAGE MEDIUM

(71) Applicant: OLYMPUS MEDICAL SYSTEMS CORP., Tokyo (JP)

(72) Inventor: Daiki ARIYOSHI, Tokyo (JP)

(73) Assignee: OLYMPUS MEDICAL SYSTEMS CORP., Tokyo (JP)

(21) Appl. No.: 19/039,273

(22) Filed: Jan. 28, 2025

Related U.S. Application Data
(60) Provisional application No. 63/626,334, filed on Jan. 29, 2024.

(51) Int. Cl. G06T 7/00; A61B 1/00; G06T 7/90; G06T 11/00

(52) U.S. Cl. CPC G06T 7/0012; A61B 1/00009; A61B 1/00045; A61B 1/00059; G06T 7/90; G06T 11/001; G06T 2207/10024; G06T 2207/10068

(57) ABSTRACT

An endoscope apparatus includes one or more processors that calculates an index of each of pixels of an image by normalizing a sum of a red pixel value and a green pixel value by a blue pixels value, selects a threshold value set corresponding to combinations of endoscope information and processor information, identifies a symptom level of each of the pixels based on the threshold value set, acquires an identification color corresponding to the symptom level of each of the pixels, and generates an identification image using the identification color of each of the plurality of pixels.

This application is based on and claims priority under 35 U.S.C. § 119 to U.S. Provisional Application No. 63/626,334, filed on Jan. 29, 2024, the entire contents of which are incorporated herein by reference.

### BACKGROUND

The present disclosure relates to an endoscope apparatus that displays an identification image according to a symptom level of an object in a body, an operation method of an endoscope apparatus that picks up an image of an object in a body and displays an identification image according to a symptom level, and a storage medium that stores a program of an endoscope apparatus that picks up an image of an object in a body and displays an identification image according to a symptom level.

International Publication No. 2018/230130 discloses an endoscope apparatus that calculates an index indicating a degree of abnormality of an object according to colors contained in an object image photographed by an endoscope, and identifies and displays the index according to a threshold value.

### SUMMARY

An endoscope apparatus of an embodiment of the present disclosure includes: one or more processors being configured to: normalize a sum of a red pixel value and a green pixel value by a blue pixel value for each of a plurality of pixels in at least a partial region of an image: calculate an index of each of the pixels: select a threshold value set according to a combination of endoscope information and processor information from a plurality of threshold value sets corresponding to a plurality of combinations of a plurality pieces of endoscope information and a plurality pieces of processor information; identify a symptom level of each of the pixels based on the selected threshold value set; acquire an identification color corresponding to the symptom level of each of the pixels; and generate an identification image using the identification color of each of the plurality of pixels.

An operation method of an endoscope apparatus of an embodiment of the present disclosure includes: performing image processing on an image pickup signal obtained by picking up an image to generate an object image; normalizing a sum of a red pixel value and a green pixel value by a blue pixel value for each of a plurality of pixels in at least a partial region of the object image; calculating an index of each of the pixels; selecting a threshold value set according to a combination of endoscope information and processor information from a plurality of threshold value sets corresponding to a plurality of combinations of a plurality pieces of endoscope information and a plurality pieces of processor information; identifying a symptom level of each of the pixels based on the selected threshold value set; acquiring an identification color corresponding to the symptom level of each of the pixels; and generating an identification image using the identification color of each of the plurality of pixels.

A storage medium of an embodiment of the present disclosure is a non-transitory computer readable storage medium configured to store a program of an endoscope apparatus. The program causes a computer to execute: performing image processing on an image pickup signal that obtained by picking up an image of an object to generate an object image; normalizing a sum of a red pixel value and a green pixel value by a blue pixel value for each of a plurality of pixels in at least a partial region of the object image; calculating an index of each of the pixels; selecting a threshold value set according to a combination of endoscope information and processor information from a plurality of threshold value sets corresponding to a plurality of combinations of a plurality pieces of endoscope information and a plurality pieces of processor information; identifying a symptom level of each of the pixels based on the selected threshold value set; acquiring an identification color corresponding to the symptom level of each of the pixels; generating an identification image using the identification color of each of the pixels; and displaying the identification image.

### BRIEF DESCRIPTION OF THE DRAWINGS

FIG. 1 is a view showing a configuration of an endoscope apparatus of an embodiment of the present disclosure.

FIG. 2 is a diagram for explaining a relationship between light absorption characteristics of blood plasma and light emission characteristics of a light source.

FIG. 3 is a view for explaining light absorption characteristics and the like of a subject.

FIG. 4 is a diagram for explaining calculation formulas of indexes of the endoscope apparatus of the embodiment of the present disclosure.

FIG. 5 is a diagram for explaining the calculation formulas of the indexes of the endoscope apparatus of the embodiment of the present disclosure.

FIG. 6 is a flowchart of an operation method of the endoscope apparatus of the embodiment of the present disclosure.

FIG. 7 is a table of a plurality of threshold value sets in the endoscope apparatus of the embodiment of the present disclosure.

FIG. 8 is a diagram showing a relationship between symptom levels and threshold values in the endoscope apparatus of the embodiment of the present disclosure.

FIG. 9 is a diagram showing a relationship between the symptom levels and identification colors in the endoscope apparatus of the embodiment of the present disclosure.

FIG. 10 is an example of a screen of a monitor in the endoscope apparatus of the embodiment of the present disclosure.

FIG. 11 shows a plurality of correction coefficient sets and a standard threshold value set in the endoscope apparatus of a modification 1 of the embodiment of the present disclosure.

### DETAILED DESCRIPTION

### <Configuration of Endoscope Apparatus>

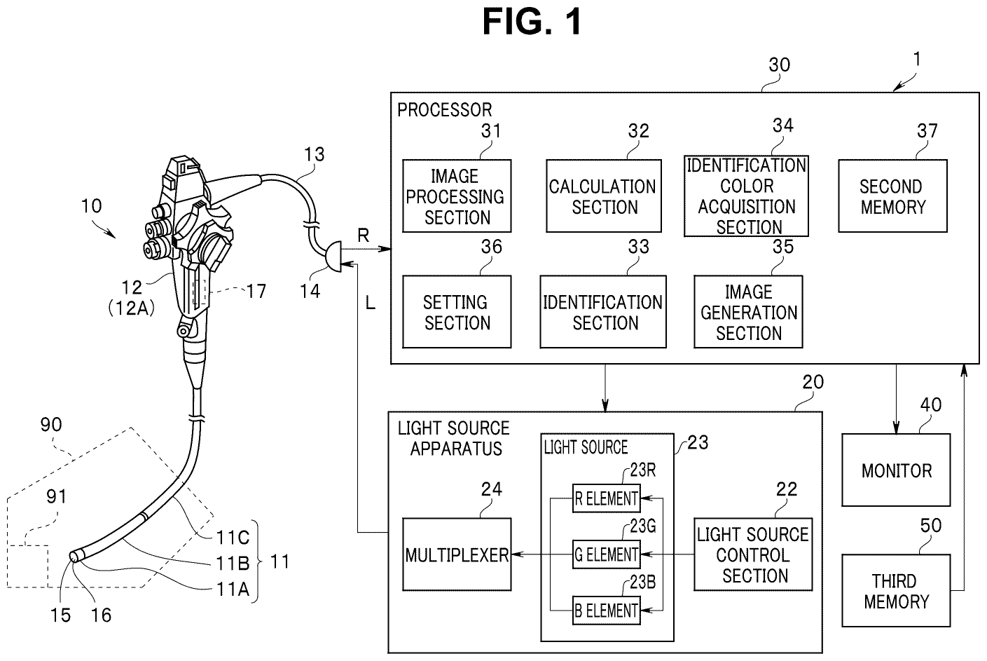

As shown in FIG. 1, an endoscope apparatus 1 of the embodiment includes an endoscope 10, a light source apparatus 20, a processor 30, a monitor 40, and a third memory 50.

The endoscope 10 includes an elongated insertion section 11 to be inserted into an examinee 90, an operation section 12 provided on a proximal end of the insertion section 11, a universal cord 13 extended from the operation section 12, and a connector 14. The operation section 12 includes a plurality of buttons 12A which are an endoscope setting section for operating an endoscope function and an image pickup function. The insertion section 11 of the endoscope 10 includes, in the following order from a distal end side, a distal end portion 11A, a bending portion 11B provided on a proximal end of the distal end portion 11A, and an elongated flexible tube 11C provided on a proximal end of the bending portion 11B. An image pickup unit 15, which is an image pickup section, and an illumination unit 16, which is an illumination section, are provided in the distal end portion 11A.

The connector 14 of the endoscope 10 is connected to the light source apparatus 20 and the processor 30. An illumination light L generated by the light source apparatus 20 is guided to the illumination unit 16 in the distal end portion 11A of the insertion section 11 to illuminate an object 91 in a body of the examinee 90. The image pickup unit 15 includes an image pickup device such as a CCD. The image pickup unit 15 converts a reflected light R from the object 91 into an electrical signal and outputs an image pickup signal of an object image to the processor 30.

The endoscope 10 includes a first memory 17 that stores endoscope information. In the present embodiment, the endoscope information is endoscope model data such as a model number of the endoscope 10. The first memory 17 is a RAM, a ROM, or an RF-ID tag, for example.

The light source apparatus 20 includes a light source control section 22, a light source 23, and a multiplexer 24.

The light source control section 22 is a light source control circuit that is connected to the light source 23 and controls the light source 23 according to a control signal from the processor 30.

The light source 23 includes a plurality of light emitting elements such as LEDs, for example. The light source 23 includes an R element 23R, a G element 23G, and a B element 23B. The R element 23R emits a red light Br in a normal band. The G element 23G emits a green light Bg in the normal band. The B element 23B emits a blue light Bb in the normal band. The B element 23B not only outputs the blue light Bb in the normal band, but also outputs a narrow-band blue light Nb by narrowing the band of the blue light with a narrow-band light filter (not shown), for example.

The multiplexer 24 multiplexes a plurality of lights inputted from the light source 23 and outputs the illumination light L to the illumination unit 16.

The processor 30 includes an image processing section 31, a calculation section 32, an identification section 33, an identification color acquisition section 34, an image generation section 35, a setting section 36, and a second memory 37. The processor 30, which is constituted of a CPU, controls the entire endoscope apparatus 1, and also generates an endoscope image based on the image pickup signal received from the endoscope 10 and generates an identification image based on the endoscope image, as described later. The second memory 37 is, for example, a RAM or a ROM that stores processor information. In the present embodiment, the processor information is processor model data such as a model number of the processor 30.

The setting section 36, which is a setting circuit, is a button or the like to which a user inputs various instructions. The setting section 36 may be a touch panel, a keyboard, a foot switch, or the buttons 12A of the endoscope 10, which is provided separately from the processor 30. For example, instructions such as a bending instruction of the bending portion, a driving instruction of the light source apparatus 20, a kind of the illumination light L that illuminates the object 91, a kind of an observation site of the object 91, and an image displayed on the monitor 40 are inputted from the setting section 36.

Note that configurations of the image processing section 31, the calculation section 32, the identification section 33, the identification color acquisition section 34, and the image generation section 35 will be described later.

At least any of the plurality of components of the processor 30, and the light source control section 22 may be configured by an internal circuit (CPU) which operates by software (programs), or may be configured by a dedicated hardware circuit.

The monitor 40 is, for example, liquid crystal, a CRT, or the like that displays a color image. The monitor 40 displays an image instructed by the processor 30. The monitor 40 having a function of a touch panel may constitute a part of the setting section 36.

The third memory 50 is a RAM, a ROM, a hard disk drive apparatus, or the like that stores data such as operation conditions, a program, or the like of the processor 30. The third memory 50 may be a non-transitory computer readable storage medium such as a CD and a DVD. The processor 30 performs predetermined processing based on the program and the data stored in the third memory 50. In addition, past examination data of the examinee 90, or the like stored on a server, for example, which is provided separately from the endoscope apparatus 1, may be transferred to the third memory 50 via the Internet or other network.

The calculation section 32 is a calculation circuit that calculates an index VI of each of a plurality of pixels of the object image outputted from the image pickup unit. The index VI quantitatively indicating a symptom level of the object 91 is calculated using predetermined calculation formulas.

The process of selecting the calculation formulas will be described below.

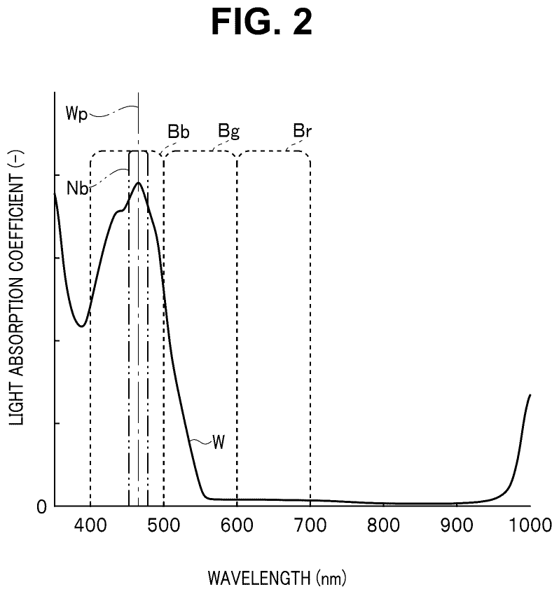

FIG. 2 is a diagram for explaining a relationship between light absorption characteristics W of blood plasma and a wavelength of a light generated by the light source 23. In FIG. 2, the red light Br in the normal band, the green light Bg in the normal band, the blue light Bb in the normal band, the narrow-band blue light Nb, the light absorption characteristics W of the blood plasma, and a peak wavelength Wp of a light absorption coefficient of the blood plasma are shown.

As shown in FIG. 2, the light absorption characteristics W of the blood plasma become low around the wavelength of 415 nm, peak around the wavelength of 465 nm, and approach 0 around the wavelength of 550 nm.

Therefore, the blue light Bb can be in the normal band, but in order to detect the blood plasma, the band of the blue light Bb may be narrowed so that a center wavelength of the blue light Bb is the same as the peak wavelength Wp of the light absorption coefficient of the blood plasma, in particular. For example, the band of the blue light Bb is narrowed so that the center wavelength is around 465 nm, and the band-narrowed blue light Bb is used as the narrow-band blue light Nb. The band of the blue light Bb may be narrowed so that the center wavelength is in a range from 460 nm to 470 nm. Furthermore, the band of the blue light Bb may be narrowed so that the center wavelength is in a range from 415 nm to 495 nm.

When irradiated with a special light including the red light Br, the green light Bg, and the narrow-band blue light Nb, the blood plasma absorbs more blue light relative to the red light and the green light, and is more yellowish than when irradiated with a normal light including the normal blue light Bb.

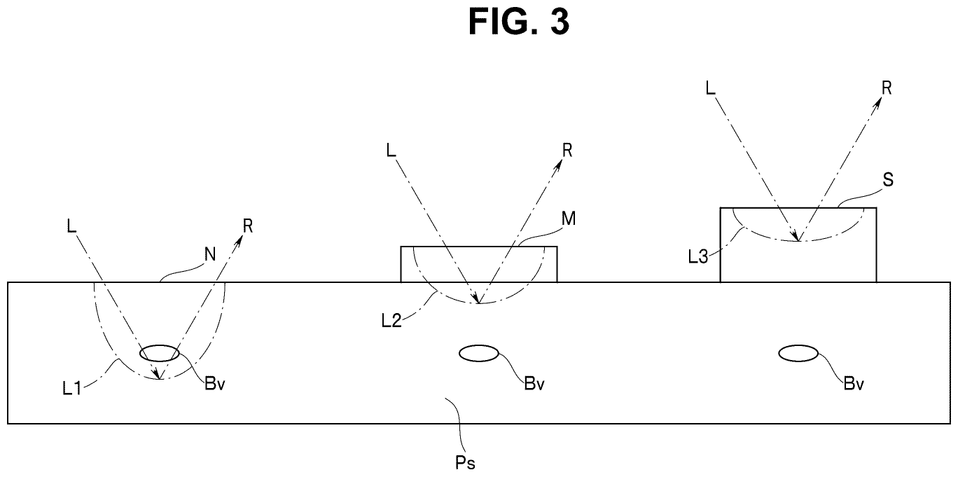

Next, FIG. 3 schematically shows a cut surface of a mucous membrane. In FIG. 3, a normal mucous membrane N, an edema M, a polyp S, blood vessels Bv, and the illumination light L are shown. Here, the illumination light L is a short-wavelength monochromatic light such as the narrow-band blue light Nb. A pigment in the mucous membrane is the blood plasma.

As shown in a light penetration region L1, a penetration degree of the illumination light L is high in the normal mucous membrane N, and the reflected light R appears pale yellow due to the pigment in the mucous membrane having the high light absorption coefficient on the short wavelength side rather than the long wavelength side.

As shown in a light penetration region L2, the penetration degree of the illumination light L is lower in the edema M than in the normal mucous membrane N. More specifically, in the edema M, the illumination light L is scattered more on the short wavelength side than on the long wavelength side due to a thickened epithelium, and is reflected without being absorbed by the pigment in the mucous membrane. Therefore, in the edema M, the reflected light R appears whiter than in the normal mucous membrane N.

As shown in a light penetration region L3, the penetration degree of the light is further lower in the polyp S than in the edema M, and the reflected light R appears further whiter than in the edema M.

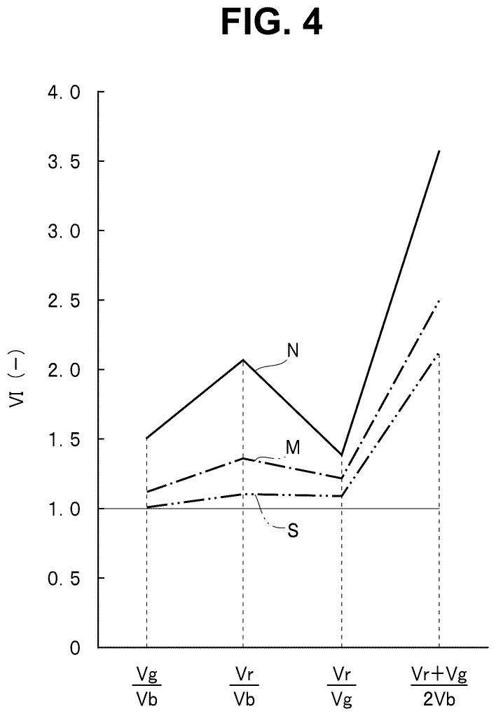

FIG. 4 shows the index VI in which a sum of a green pixel value Vg, a red pixel value Vr, and a blue pixel value Vb, or a sum of the green pixel value Vg and the red pixel value Vr, of each pixel contained in the endoscope image is normalized. A pixel value V is acquired as 8-bit data (0 to 255), for example.

FIG. 4 shows the differences in the indexes VI due to the differences in the calculation formulas of the indexes VI among the normal mucous membrane N, the edema M, and the polyp S. In FIG. 4, each of “Vg/Vb”, “Vr/Vb”, “Vr/Vg”, and “(Vr+Vg)/2Vb” on the X-axis indicates a calculation formula for the index VI, and the Y-axis indicates the index VI normalized by each of the calculation formulas.

The solid line indicates the normal mucous membrane N, the dotted line indicates the edema M, and the double-dotted line indicates the polyp S. Hereafter, the edema M and the polyp S are referred to as abnormal mucous membranes.

In the mucous membrane in the body, for example, the mucous membrane of the nasal and paranasal sinuses, the severity of the symptom level increases in the order of the normal mucous membrane N, the edema M, and the polyp S. There is a difference in color between the normal mucous membrane N and the abnormal mucous membrane, and as the severity of the symptom level increases, the epithelium of the mucous membrane thickens and the whiteness of the appearance also increases. Therefore, the calculation formula that gives the greatest values for the index VI of the normal mucous membrane N and the index VI of the polyp S is “(Vr+Vg)/2 Vb”.

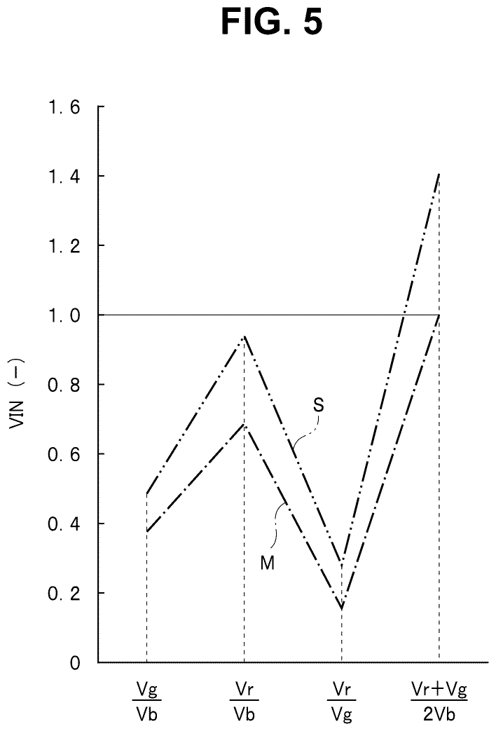

FIG. 5 shows indexes VIN of the edema M and the polyp S in which the indexes VI of the edema M and the polyp S, which are the same as those in FIG. 4, are normalized by the index VI of the normal mucous membrane N. In FIG. 5, the X-axis indicates the calculation formulas for the index VIN used to calculate the index VIN, which is normalized by the index VI of the normal mucous membrane N, and the Y-axis indicates the index VIN.

As shown in FIG. 4 and FIG. 5, in the normal mucous membrane N and the polyp S, the indexes VI and VIN calculated by the calculation formula “(Vr+Vg)/2Vb” is larger than the indexes VI and VIN calculated by other calculation formulas for the index.

In other words, the index VIN calculated by the calculation formula “(Vr+Vg)/2Vb” shows the large difference between the color of the normal mucous membrane N and that of the abnormal mucous membrane.

### <Operation Method of Endoscope Apparatus>

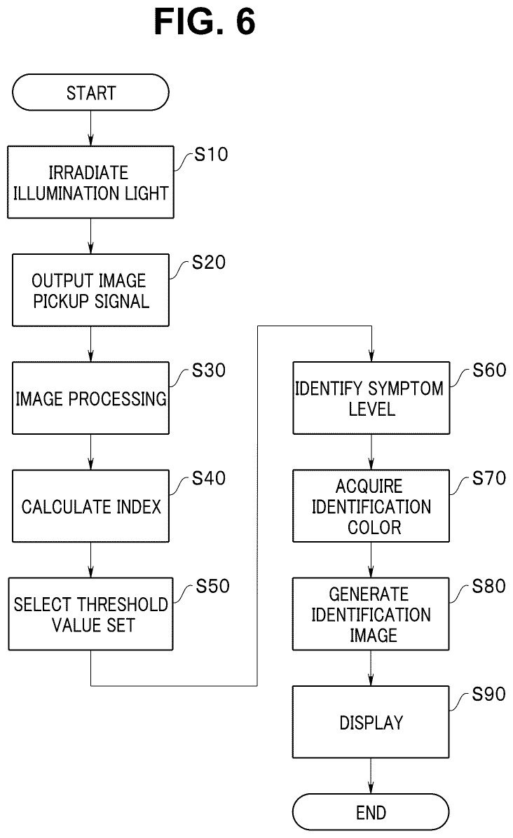

An operation method of the endoscope apparatus 1 will be described using a flowchart of FIG. 6.

### <Step S10> Irradiation of Illumination Light

The insertion section 11 of the endoscope 10 is inserted into a living body of the examinee 90, for example, into the nasal sinuses. The illumination light L from the light source apparatus 20 is irradiated onto the mucous membrane, which is the object 91, via the illumination unit 16 in the distal end portion 11A. The illumination light L includes the red light Br, the green light Bg, and the blue light Bb.

### <Step S20> Output of Image Pickup Signal

The image pickup unit 15 in the distal end portion 11A receives the reflected light R from the object 91, converts the received reflected light R into an electrical signal, and outputs an image pickup signal to the processor 30.

### <Step S30> Image Processing

The image processing section 31 is an image processing circuit that performs image processing such as a gain adjustment, a white balance adjustment, a gamma correction, a contour enhancement correction, and a scaling adjustment, for example, based on the image pickup signal, to generate an endoscope image which is the object image.

### <Step S40> Calculation of Index

The calculation section 32 calculates the index VI of each of the plurality of pixels of the object image by normalizing, for each of the pixels, the sum of the red pixel value Vr and the green pixel value Vg by a value which is twice of the blue pixel value Vb (Nb). In other words, the calculation section 32 calculates the indexes VI of the plurality of regions (pixels) of the object.

In the examples shown in FIG. 4 and FIG. 5, “(Vr+Vg)/2Vb” is used as a calculation formula that normalizes the pixel values and calculates the index VI. However, the calculation formula can be changed appropriately if a calculation formula normalizes the sum of the red pixel value and the green pixel value by a value which is twice of the blue pixel value.

For example, the index VI may be converted into 8-bit data (0 to 255), a numerical value may be further added to the 8-bit data, or a value of k in the calculation formula “(Vr+Vg)/kVb” may be changed. In the following, the index VI is calculated using a formula 1.

$$VI = 32 \times {log}_{2} [ ( Vr + Vg ) / 2 Vb ] + 256 \qquad 〈 Formula 1 〉$$

The calculation section 32 may calculate the index VI using any of a plurality of calculation formulas corresponding to each of the plurality of objects 91 (nasal and paranasal sinuses, digestive tract, for example).

Note that the calculation section 32 may divide the object image into areas constituted of a plurality of pixels (for example, pixels in units of 25=5×5) and calculate the index VI for each area. In other words, the calculation section 32 may calculate the index based on an average value of the pixel values of the plurality of pixels included in each of the areas.

The each of the areas may overlap with the adjacent area in some pixels (for example, 16 pixels on the outer periphery of the area of 25=5×5 pixels) each other.

### <Step S50> Selection of Threshold Value Set

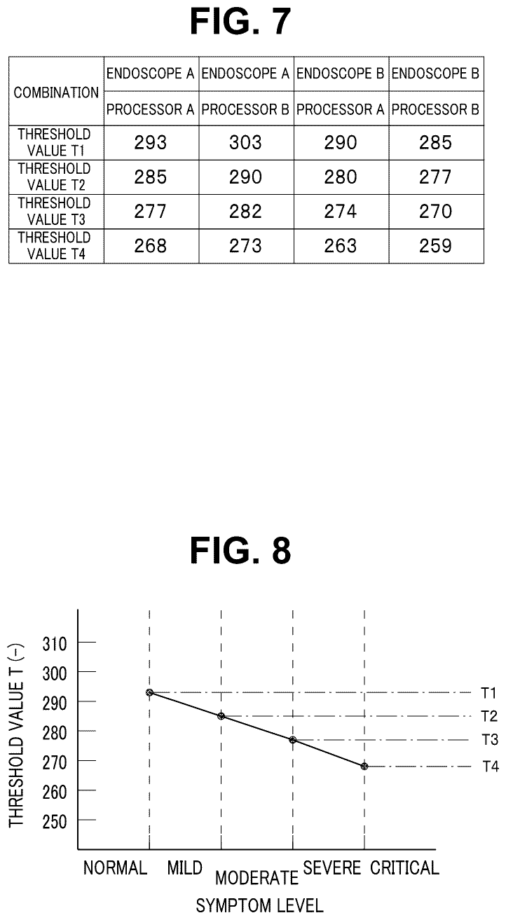

The identification section 33 is an identification circuit that selects a threshold value set TS to be used in identification among a plurality of threshold value sets TS (FIG. 7), and identifies the symptom level of each of the pixels using the selected threshold value set TS, based on the index VI.

In the endoscope apparatus 1, the symptom levels include five levels of “normal, mild, moderate, severe, and critical”. If there are three or more symptom levels, it is easier to determine a detailed symptom than when there are only two levels of “normal and abnormal”.

To identify the five symptom levels, four threshold values T (a first threshold value T1 to identify between normal and mild, a second threshold value T2 to identify between mild and moderate, a third threshold value T3 to identify between moderate and severe, and a fourth threshold value T4 to identify between severe and critical) can be used.

The identification section 33 selects the threshold value set TS to be used in identification from a table of the plurality of threshold value sets TS (FIG. 7) according to a plurality of combinations of a plurality of endoscopes and a plurality of processors stored in the second memory 37 or the third memory 50. The table of the threshold value sets TS is set appropriately in advance, based on determination by a plurality of intellects, or the like.

The identification section 33 acquires the endoscope information of the endoscope 10 connected, from the first memory 17 of the endoscope 10 via a wired or wireless connection. The endoscope information may be inputted by a user using the setting section 36 of the processor 30. The processor information of the processor 30 is stored in the second memory 37, for example.

### <Step S60> Identification of Symptom Level

FIG. 8 shows an example of the plurality of threshold values T in a case where the endoscope 10 is an endoscope A and the processor 30 is a processor A (FIG. 7). The identification section 33 identifies the symptom level of each of the pixels using the selected threshold value set TS, based on the index VI calculated by the calculation section 32.

### <Step S70> Identification Color Acquisition Section

The identification color acquisition section 34 acquires an identification color according to the symptom level of the pixel acquired by the identification section 33.

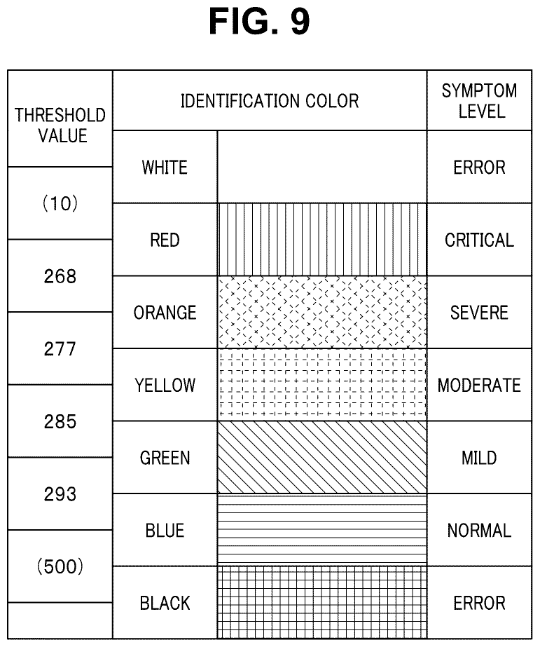

FIG. 9 shows the identification colors according to the symptom levels. The index VI is data in a range obtained by adding 256 to the 8-bit data (0 to 511). The plurality of threshold values T and the identification colors are stored in the second memory 37.

Note that in the example of FIG. 9, the identification color acquisition section 34 acquires a plurality of colors with different hues, but may acquire a plurality of saturations with different clearness, a plurality of luminosities with different brightnesses, a plurality of hatching patterns with different intervals, or a plurality of designs with different patterns, or the like.

Note that the object image may include a pixel having an error pixel value of a color which is not normally generated in photography. In the endoscope apparatus 1, a pixel having a pixel value V, which is equal to or less than a predetermined lower limit pixel value or is equal to or more than a predetermined upper limit pixel value, among at least one of the red pixel value, the green pixel value, and the blue pixel value, is a first error pixel. For example, in pixels having the pixel values V within a range of (0 to 255), a pixel having the pixel value of 5 or less, or 250 or more is the first error pixel.

Furthermore, a pixel having the index VI which is equal to or less than a predetermined lower limit threshold value or equal to or more than a predetermined upper limit threshold value is a second error pixel. For example, in the example shown in FIG. 9, a pixel having the index VI which is the lower limit threshold value of 10 or less, or the upper limit threshold value of 500 or more is the second error pixel.

The identification color acquisition section 34 acquires an error color for the error pixel (the first error pixel or the second error pixel). The image generation section generates the identification image using the error color for the error pixel. The error color is not limited to white and black illustrated in FIG. 9, but may be also gray or other colors, for example. Also, pixels having the threshold values or the like which are equal to or less than the lower limit or equal to or more than the upper limit may be the same error color. The numerical value of criteria for determining the error pixel and data on the error color are stored in the second memory 37 or the third memory 50 in advance.

### <Step S80> Generation of Identification Image

The identification color acquisition section 34 acquires the identification color corresponding to the symptom level of each of the pixels. The image generation section 35 is an identification color acquisition circuit that generates the identification image using the identification color of each of the plurality of pixels.

### <Step S90> Display

The monitor 40 displays the identification image.

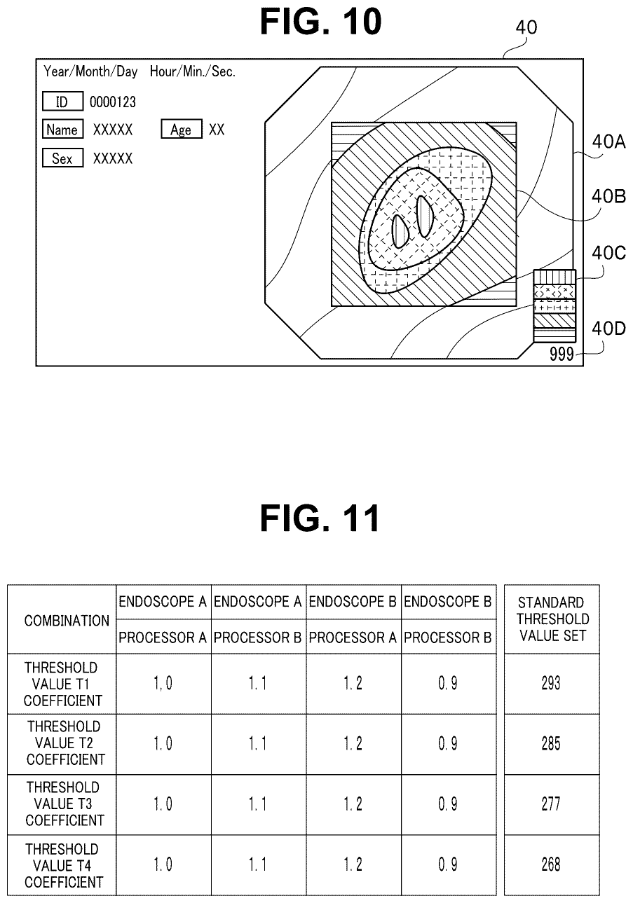

FIG. 10 shows an example of a display image on the monitor 40. In FIG. 10, a partial region of an endoscope image 40A that is displayed in color is replaced and displayed as an identification image 40B. In other words, a superimposed image in which the identification image 40B is superimposed on the endoscope image 40A is displayed.

On the monitor 40, an average value 40D of the indexes is displayed with an identification color list display 40C. In other words, the calculation section 32 calculates the average value 40D of the indexes of the plurality of pixels, and the monitor 40 displays the average value 40D of the indexes.

The user can easily understand a symptom of the examinee based on the average value 40D of the indexes.

The monitor 40 may display only the identification image 40B. In other words, the calculation section 32 may calculate the indexes from the plurality of pixels in the entire region of the endoscope image 40A which is the object image, and the image generation section 35 may generate the identification image corresponding to the entire region of the endoscope image 40A.

The endoscope image 40A may be displayed in a main region of the monitor 40, and the identification image 40B in a region surrounded by a frame in the endoscope image 40A may be displayed in a region other than the endoscope image 40A. At least one of a position and a range (area) of a partial region where the identification image 40B is generated in the entire region of the endoscope image 40A, which is the object image, can be appropriately selected by operating the setting section 36.

In a narrow conduit, it may not be easy to direct a center of the endoscope image 40A (a center of a field of view of the image pickup unit 15) to an interested region. However, by selecting at least one of the position and the range of the region where the identification image 40B is displayed, the user can easily identify the interested region.

The endoscope apparatus 1 of the present embodiment can display the appropriate identification image according to the combination of the endoscope and the processor. In the present embodiment, the threshold value is determined according to the combination of the endoscope and the processor, but the threshold value may be determined according to a combination with the light source apparatus or the monitor in the endoscope apparatus. In this case, a memory that stores model information is provided in the light source apparatus or the monitor, and the information is read out through a connection line.

As described above, the operation method of the endoscope apparatus of the embodiment includes performing image processing on an image pickup signal obtained by picking up an image of an object in a body of an examinee to generate an object image, calculating an index of each of a plurality of pixels in at least a partial region of the object image by normalizing, for each of the pixels, a sum of a red pixel value and a green pixel value by a value which is twice of a blue pixel value, selecting a threshold value set according to a combination of endoscope information and processor information from a plurality of threshold value sets corresponding to a plurality of combinations of the plurality pieces of endoscope information and a plurality pieces of processor information, identifying a symptom level of each of the pixels as one of a plurality of symptom levels using the selected threshold value set, acquiring an identification color corresponding to the symptom level of each of the pixels, generating an identification image using the identification color of each of the plurality of pixels, and displaying the identification image.

A program of another embodiment causes a computer to execute the above processing.

A storage medium of another embodiment is a non-transitory computer readable storage medium, and stores a program that causes a computer to execute the above processing.

### <Modifications>

Endoscope apparatuses 1A to 1C of modifications are similar to the endoscope apparatus 1 of the embodiment, and have the same effects as the endoscope apparatus 1. Therefore, components having the same functions as those of the endoscope apparatus 1 are denoted by the same reference signs and the explanation thereof will be omitted.

### <Modification 1>

The endoscope apparatus 1A of the present modification acquires a threshold value set TS using a correction data set selected from a table of correction data sets of a plurality of threshold values according to combinations of a plurality pieces of endoscope information and a plurality pieces of processor information, and a standard threshold value set (FIG. 11). The correction data sets and the standard threshold value set are stored in the second memory 37 or the third memory 50, for example.

For example, when the endoscope 10 is an endoscope B and the processor 30 is a processor B, a first threshold value T1 is 290 (=296×0.98),

The endoscope apparatus 1A has a smaller capacity for data stored in the second memory 37 or the like than that of the endoscope apparatus 1.

### <Modification 2>

Even if the endoscopes 10 or the processors 30 are the same model manufactured to the same specification, there are individual differences.

In the endoscope apparatus 1B of the present modification, endoscope information includes endoscope model data such as a model number and endoscope individual data such as a manufacturing number, of the endoscope 10. Processor information includes processor model data such as a model number and processor individual data such as a manufacturing number, of the processor 30.

The endoscope individual data such as the manufacturing number of the endoscope may include information on the endoscope model data such as the model number. Similarly, the processor individual data such as the manufacturing number of the processor may include information on the processor model data such as the model number.

Therefore, in the endoscope apparatus 1B, the endoscope information may be the endoscope individual data, and the processor information may be the processor individual data.

For example, the endoscope individual data is a correction coefficient stored in the first memory 17 after manufacturing and at a shipping inspection of the endoscope 10. The processor individual data is a correction coefficient stored in the second memory 37 after manufacturing and at a shipping inspection of the processor 30.

For example, in the table shown in FIG. 7 or FIG. 11, when the endoscope 10 is the endoscope A and the correction coefficient of the endoscope individual data is 0.99, and the processor 30 is the processor A and the correction coefficient of the processor individual data is 1.02, the first threshold value T1 is 266 (=293×0.99×1.02).

In the endoscope apparatus 1B, even if there are the individual differences in the endoscope or the processor, an appropriate identification image can be displayed.

### <Modification 3>

A color tone or the like of the endoscope image may be changed when a user makes adjustments by the setting section 36. In the endoscope apparatus 1C of the present modification, the threshold value set TS is corrected according to a change of a parameter of the image processing.

For example, when a red level is adjusted by +5%, the processor 30 multiplies all the threshold values T of the threshold value set TS by the correction coefficient of 1.10. Conversely, when the red level is adjusted by −5%, the processor 30 multiplies all the threshold values T of the threshold value set TS by the correction coefficient of 0.92.

In the endoscope apparatus 1C, even if the endoscope image is adjusted by a user, an appropriate identification image can be displayed.

According to the embodiment of the present disclosure, an endoscope apparatus that displays an appropriate identification image according to a combination of an endoscope and a processor, an operation method of an endoscope apparatus that displays an appropriate identification image according to a combination of an endoscope and a processor, and a storage medium that stores a program of an endoscope apparatus that displays an appropriate identification image according to a combination of an endoscope and a processor can be provided.

Note that the above described scope of a numerical value, for example, a wavelength is not limited to the above described range and can be increased or decreased appropriately. Also, the endoscope 10 may be a rigid endoscope in which the insertion section 11 is rigid. The present disclosure is not limited to the above explained embodiment and the like, and various modifications and alternations can be made in a scope not departing from the gist of the present disclosure.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, performs one or more of the methods described above.

### Example

1. An endoscope apparatus comprising:
   an endoscope configured to pick up an image of an object in a body of an examinee and output an image pickup signal;
   a processor that performs image processing on the image pickup signal to generate an object image, the processor being configured to:
      calculate an index of each of a plurality of pixels in at least a partial region of the object image by normalizing, for each of the pixels, a sum of a red pixel value and a green pixel value by a blue pixel value:
      select a threshold value set according to a combination of endoscope information and processor information from a plurality of threshold value sets corresponding to a plurality of combinations of a plurality pieces of endoscope information and a plurality pieces of processor information;
      identify a symptom level of each of the pixels as one of a plurality of symptom levels using the selected threshold value set;
      acquire an identification color corresponding to the symptom level of each of the pixels; and
      generate an identification image using the identification color of each of the plurality of pixels; and
   a monitor configured to display the identification image.

What is claimed is:

1. An endoscope apparatus comprising:
one or more processors being configured to:
normalize a sum of a red pixel value and a green pixel value by a blue pixel value for each of a plurality of pixels in at least a partial region of an image;
calculate an index of each of the pixels;
select a threshold value set according to a combination of endoscope information and processor information from a plurality of threshold value sets corresponding to a plurality of combinations of a plurality pieces of endoscope information and a plurality pieces of processor information;
identify a symptom level of each of the pixels based on the selected threshold value set;
acquire an identification color corresponding to the symptom level of each of the pixels; and
generate an identification image using the identification color of each of the pixels.

2. The endoscope apparatus according to claim 1, wherein
the endoscope information is at least one of endoscope model data and endoscope individual data, and
the processor information is at least one of processor model data or processor individual data.

3. The endoscope apparatus according to claim 2, wherein
the plurality of threshold value sets include a plurality of correction data sets according to the plurality of combinations of the plurality pieces of endoscope information and the plurality pieces of processor information, and a standard threshold value set.

4. The endoscope apparatus according to claim 2, wherein
the one or more processors is configured to correct the threshold value set according to a change of a parameter of an image processing.

5. The endoscope apparatus according to claim 1, further comprising
the monitor configured to display the identification image.

6. The endoscope apparatus according to claim 1, wherein
the one or more processors being configured to calculate the index by normalizing a sum of a red pixel value and a green pixel value by a value which is twice of a blue pixel value.

7. An operation method of an endoscope apparatus comprising:
performing image processing on an image pickup signal obtained by picking up an image of an object to generate an object image;
normalizing a sum of a red pixel value and a green pixel value by a blue pixel value for each of a plurality of pixels in at least a partial region of the object image;
calculating an index of each of the pixels;
selecting a threshold value set according to a combination of endoscope information and processor information from a plurality of threshold value sets corresponding to a plurality of combinations of a plurality pieces of endoscope information and a plurality pieces of processor information;
identifying a symptom level of each of the pixels based on the selected threshold value set;
acquiring an identification color corresponding to the symptom level of each of the pixels; and
generating an identification image using the identification color of each of the pixels.

8. The operation method of the endoscope apparatus according to claim 7, wherein
the endoscope information is at least one of endoscope model data and endoscope individual data, and
the processor information is at least one of processor model data and processor individual data.

9. A non-transitory computer readable storage medium configured to store a program of an endoscope apparatus,
the program causing a computer to execute:
performing image processing on an image pickup signal obtained by picking up an image of an object to generate an object image;
normalizing a sum of a red pixel value and a green pixel value by a blue pixel value for each of a plurality of pixels in at least a partial region of the object image;
calculating an index of each of the pixels;
selecting a threshold value set according to a combination of endoscope information and processor information from a plurality of threshold value sets corresponding to a plurality of combinations of a plurality pieces of endoscope information and a plurality pieces of processor information;
identifying a symptom level of each of the pixels based on the selected threshold value set;
acquiring an identification color corresponding to the symptom level of each of the pixels;
generating an identification image using the identification color of each of the pixels; and
displaying the identification image.

10. The endoscope apparatus according to claim 1, further comprising:
an endoscope configured to pick up the image and output an image pickup signal.

11. The endoscope apparatus according to claim 1, wherein
the one or more processors is configured to control a monitor to display the identification image.

12. The operation method according to claim 7, wherein
the endoscope information is at least one of endoscope model data and endoscope individual data, and
the processor information is at least one of processor model data or processor individual data.

13. The operation method according to claim 12, wherein
the plurality of threshold value sets include a plurality of correction data sets according to the plurality of combinations of the plurality pieces of endoscope information and the plurality pieces of processor information, and a standard threshold value set.

14. The operation method according to claim 12, comprising:
correcting the threshold value set according to a change of a parameter of an image processing.

15. The endoscope apparatus according to claim 7, comprising:
calculating the index by normalizing a sum of a red pixel value and a green pixel value by a value which is twice of a blue pixel value.

16. The endoscope apparatus according to claim 7, comprising:
displaying the identification image.

17. The non-transitory computer readable storage medium according to claim 9, wherein
the endoscope information is at least one of endoscope model data and endoscope individual data, and
the processor information is at least one of processor model data or processor individual data.

18. The non-transitory computer readable storage medium according to claim 17, wherein
the plurality of threshold value sets include a plurality of correction data sets according to the plurality of combinations of the plurality pieces of endoscope information and the plurality pieces of processor information, and a standard threshold value set.

19. The non-transitory computer readable storage medium according to claim 17, comprising:
correcting the threshold value set according to a change of a parameter of an image processing.

20. The non-transitory computer readable storage medium according to claim 9, comprising:
calculating the index by normalizing a sum of a red pixel value and a green pixel value by a value which is twice of a blue pixel value.
